(19) United States
(12) Patent Application Publication
(10) Pub. No.: US 2026/0090911 A1
(43) Pub. Date: Apr. 2, 2026

(54) OPHTHALMIC INSERTION TOOL INCLUDING INTEGRAL SCLERAL MARKER

(71) Applicant: Bausch + Lomb Ireland Limited, Dublin (IE)

(72) Inventor: James T. PERKINS, St. Peters, MO (US)

(21) Appl. No.: 18/903,180

(22) Filed: Oct. 1, 2024

(51) Int. Cl. A61F 9/00; A61B 17/34

(52) U.S. Cl. CPC A61F 9/0008; A61B 17/3421

(57) ABSTRACT

An ophthalmic insertion tool includes a housing having a distal end and a proximal end, and a cannula attached to the distal end of the housing. The cannula includes a distal tip with at least one port in communication with a lumen extending through the cannula. The tool includes a retractable sheath coupled with the distal end of the housing. The retractable sheath is configured to move between a first position covering the distal tip of the cannula and a second position exposing the distal tip of the cannula. The tool includes at least one scleral marker integral with the housing. The at least one scleral marker is located adjacent the distal end of the housing, the proximal end of the housing, or both the distal end and the proximal end.

### FIELD

The present disclosure relates to ophthalmic insertion tools, particularly to ophthalmic insertion tools including scleral markers which are integral with a housing of the ophthalmic insertion tool.

### BACKGROUND

This section provides background information related to the present disclosure which is not necessarily prior art.

Ophthalmic insertion tool devices including cannulas are used in posterior retina surgery to gain access to the posterior part of the eye. In modern posterior surgery, three entry sites or incisions (e.g., sclerotomies) are typically created for infusion, illumination, and use of various tools to address different issues with the retina.

For example, a marker may be placed at the rear of an ophthalmic insertion tool (e.g., an entry site alignment (ESA) device) where a surgeon has to rotate or flip the device around in order to mark the sclera. The surgeon then removes a sheath, which may be placed on a tray, in order to insert a trocar and cannula into the sclera. Once the cannula is placed into the sclera, the surgeon replaces the sheath on the ESA device. These multiple steps may increase the risk of inadvertent damage to the trocar tip, and the surgeon being cut by the trocar tip. Some trocar handles include longitudinal grooves to help inhibit or prevent the cap from damaging the tip when the cap/sheath is removed, but tip damage may still occur if a nurse or surgeon is not careful.

It would be desirable to provide an irrigation sleeve that is soft enough to sufficiently collapse to create a seal with eye incision but yet hard enough to not collapse upon insertion through the eye incision.

### SUMMARY

This section provides a general summary of the disclosure, and is not a comprehensive disclosure of its full scope or all of its features.

According to one aspect of the present disclosure, an ophthalmic insertion tool includes a housing having a distal end and a proximal end, and a cannula attached to the distal end of the housing. The cannula includes a distal tip with at least one port in communication with a lumen extending through the cannula. The tool includes a retractable sheath coupled with the distal end of the housing. The retractable sheath is configured to move between a first position covering the distal tip of the cannula and a second position exposing the distal tip of the cannula. The tool includes at least one scleral marker integral with the housing. The at least one scleral marker is located adjacent the distal end of the housing, the proximal end of the housing, or both the distal end and the proximal end.

According to another aspect of the present disclosure, an ophthalmic insertion tool package includes a container package, and at least one ophthalmic insertion tool inside the container package. The ophthalmic insertion tool includes a housing having a distal end and a proximal end, and a retractable sheath coupled with the distal end of the housing. The retractable sheath is configured to move between a first position covering the distal end of the housing and a second position exposing the distal end of the housing. The ophthalmic insertion tool includes at least one scleral marker integral with the housing, the at least one scleral marker located adjacent the distal end of the housing, the proximal end of the housing, or both the distal end and the proximal end. At least one cannula is inside the container package, the cannula including a distal tip with at least one port in communication with a lumen extending through the cannula.

Further aspects and areas of applicability will become apparent from the description provided herein. It should be understood that various aspects and features of this disclosure may be implemented individually or in combination with one or more other aspects or features. It should also be understood that the description and specific examples herein are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an orthogonal view of an ophthalmic insertion tool including an integral scleral marker, according to an example embodiment;

FIG. 2 is an orthogonal view of the ophthalmic insertion tool of FIG. 1 in a retracted position;

FIG. 3 is a close up view of the exposed trocar tip of the ophthalmic insertion tool of FIG. 2;

FIG. 4 is a sectional view of the exposed trocar tip of FIG. 3;

FIG. 5 is a side view of a sheath of an ophthalmic insertion tool in an extended position;

FIG. 6 is a side view of the sheath of FIG. 5 in a retracted position; and

FIG. 7 is a flow chart depicting an example process for using an ophthalmic insertion tool.

Corresponding reference numerals indicate corresponding parts throughout the several views of the drawings.

### DETAILED DESCRIPTION

Example embodiments will now be described more fully with reference to the accompanying drawings.

Ophthalmic insertion tool devices including cannulas are used in posterior retina surgery to gain access to the posterior part of the eye. In modern posterior surgery, three entry sites or incisions (e.g., sclerotomies) are typically created for infusion, illumination, and use of various tools to address different issues with the retina.

For example, a marker may be placed at the rear of an ophthalmic insertion tool (e.g., an entry site alignment (ESA) device) where a surgeon has to rotate or flip the device around in order to mark the sclera. The surgeon then removes a sheath, which may be placed on a tray, in order to insert a trocar and cannula into the sclera. Once the cannula is placed into the sclera, the surgeon replaces the sheath on the ESA device. These multiple steps may increase the risk of inadvertent damage to the trocar tip, and the surgeon being cut by the trocar tip. Some trocar handles include longitudinal grooves to help inhibit or prevent the cap from damaging the tip when the cap/sheath is removed, but tip damage may still occur if a nurse or surgeon is not careful.

In some example embodiments, an integrated retractable sheath allows a surgeon to keep the ophthalmic insertion tool assembly in on position in their hand in order to mark the sclera, and then pull back the sheath with their free hand to insert the cannula into the patient's sclera. Once the cannula has been inserted, the sheath is pushed back into the extended position to protect the sharp trocar blade. This reduces or minimizes the number of steps needed to insert the cannula into the patient's sclera.

The sheath may include one or more snap features that retain or lock the sheath in the extended position with enough retention force to allow the surgeon to use the markers (e.g., integral scleral markers located at an end of the sheath) to create indentation marks at a precise location from the cornea on the sclera. Once the sclera is marked, the sheath and scleral marker may be pulled back into a retracted position to allow placement of the cannula.

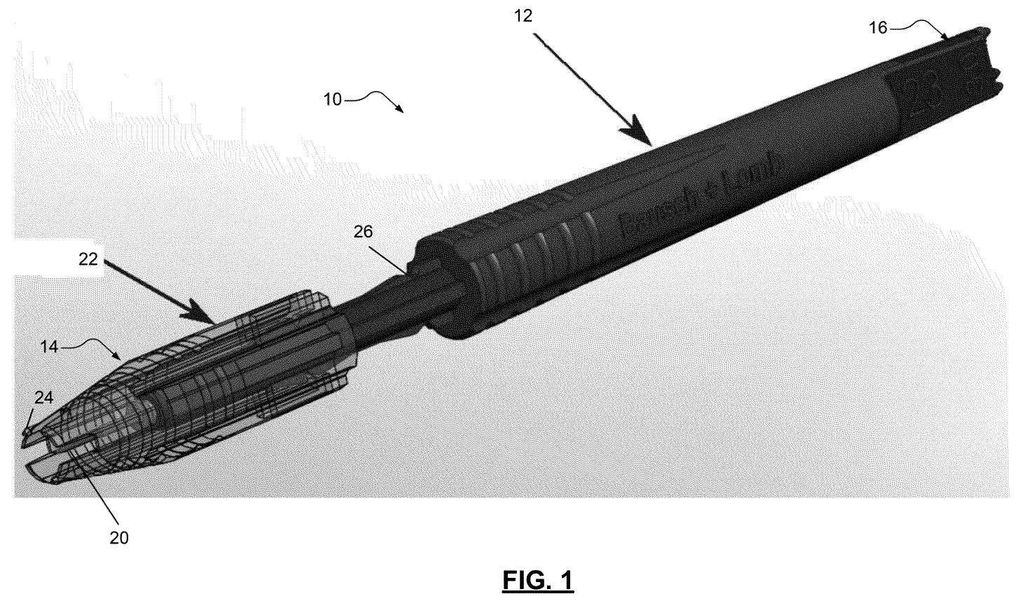

FIG. 1 is an orthogonal view of an ophthalmic insertion tool 10 including an integral scleral marker, according to an example embodiment. The ophthalmic insertion tool 10 includes a housing 12 having a having a distal end 14 and a proximal end 16.

At least one scleral marker 24 is integral with the housing 12. The at least one scleral marker 24 may be located adjacent the distal end 14 of the housing 12, as shown in FIG. 1. For example, the scleral marker 24 may include a protrusion from, e.g., the retractable sheath 22 of the housing 12, such as a pointed tip which will leave an indentation in the sclera but not incise the sclera when the scleral marker 24 is pressed against the sclera. In other example embodiments, the scleral marker(s) 24 may be located at the proximal end 16 of the housing 12, or may be located at both the distal end 14 and the proximal end 16.

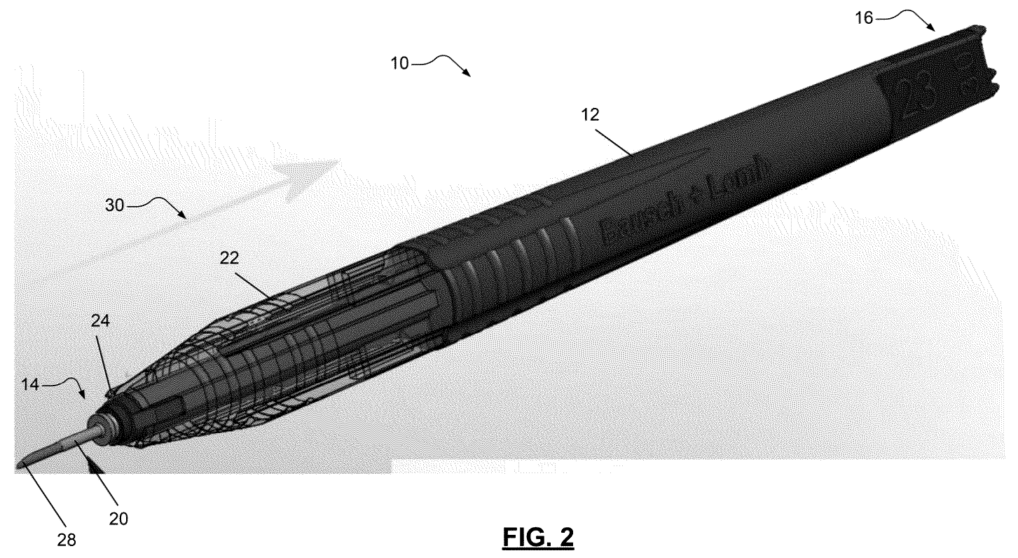

A retractable sheath 22 is coupled with the distal end 14 of the housing. FIG. 2 is an orthogonal view of the ophthalmic insertion tool 10 of FIG. 1, where the retractable sheath 22 is in a retracted position. For example, the ophthalmic insertion tool 10 includes a cannula 20 is attached to the distal end 14 of the housing 12.

The cannula 20 includes a distal tip 28 with at least one port in communication with a lumen extending through the cannula 20. The distal tip 28 of the cannula is a sharp tip (e.g., a needle) configured to puncture a posterior of an eye of a patient.

The retractable sheath 22 is configured to move between a first position covering the distal tip 28 of the cannula 20 (as shown in FIG. 1), and a second position exposing the distal tip 28 of the cannula 20 (as shown in FIG. 2). For example, as shown in FIG. 2, the retractable sheath 22 may be configured to move in a longitudinal direction 30 along an outer surface of the housing 12 between the first position and the second position.

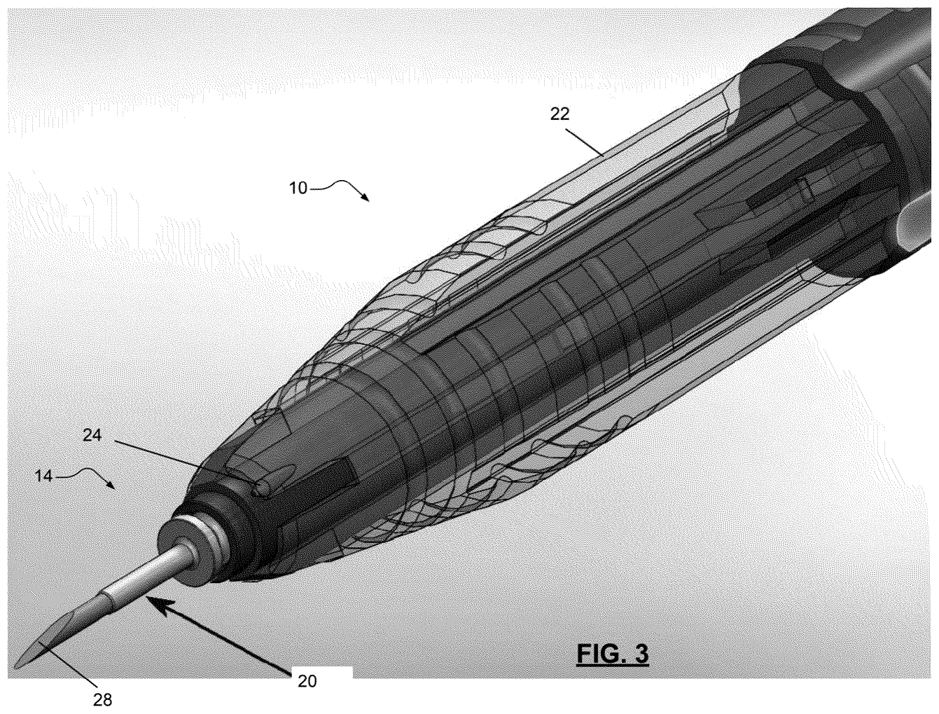

In some example embodiments, the retractable sheath 22 is configured to retract from the first position to the second position in response to the distal end 14 of the housing 12 pressing against a sclera of an eye of a patient. FIG. 3 is a close up view of the exposed trocar tip 28 (e.g., the distal tip) of the ophthalmic insertion tool 10. As shown in FIG. 3, the retractable sheath 22 is in a retracted position, exposing the trocar tip 28 for puncturing a posterior of an eye of a patient.

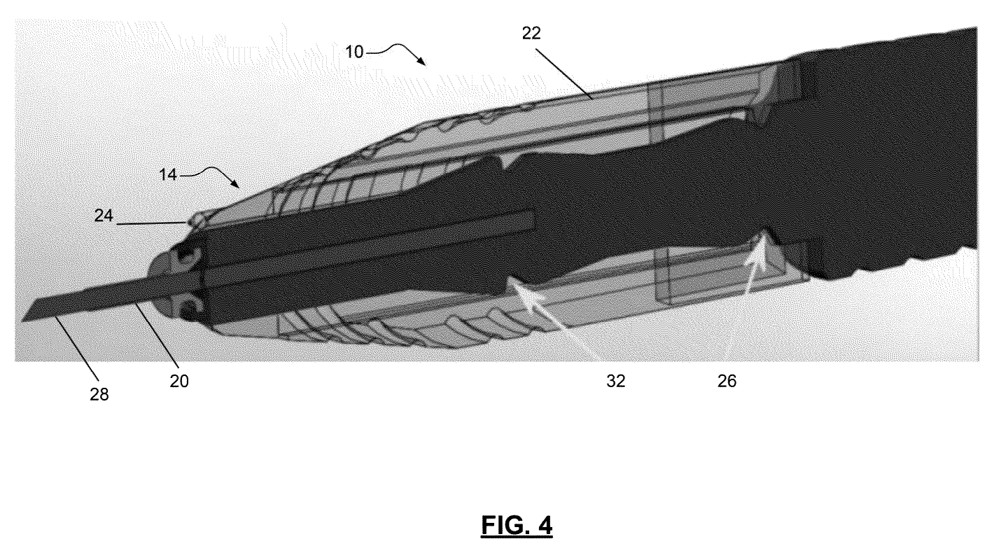

FIG. 4 is a sectional view of the exposed trocar tip 28 of FIG. 3. As shown in FIG. 4, the retractable sheath 22 is in a retracted position. The 12 may include a first locking point 26 configured to maintain the retractable sheath 22 in the second position (e.g., a retracted position), to expose the distal tip 28 of the cannula 20 to facilitate puncturing the sclera with the distal tip 28. For example, the locking point 26 may include an indentation that receives a lip, protrusion, etc. of the retractable sheath 22 to maintain the retractable sheath 22 in the retracted position, until a sufficient force is applied to the retractable sheath 22 to move the retractable sheath back to the extended position covering the cannula 20.

As shown in FIG. 4, the housing includes a second locking point 32 configured to maintain the retractable sheath 22 in the first position (e.g., the extended position) to protect the distal tip 28 of the cannula 20 from damage. The second locking point 32 is configured to apply a retention force to the retractable sheath 22 to facilitate marking the sclera according to the at least one scleral marker 24, while the retractable sheath 22 is maintained in the first position.

For example, the locking point 32 may include an indentation that receives a lip, protrusion, etc. of the retractable sheath 22 to maintain the retractable sheath 22 in the extended position, until a sufficient force is applied to the retractable sheath 22 to move the retractable sheath back to the retracted position exposing the cannula 20. The force needed to retract the retractable sheath 22 may be greater than a force that is applied while marking the sclera using a scleral marker 24 (e.g., simply using the scleral marker 24 may not provide enough force to overcome the retention of the second locking point 32).

In some example embodiments, the housing 12 is configured to release the cannula 20 to maintain the cannula in the sclera, as the housing 12 is moved away from the eye of the patient. The cannula 20 may be configured to facilitate insertion of at least one surgical instrument into the eye of the patient through the cannula 20.

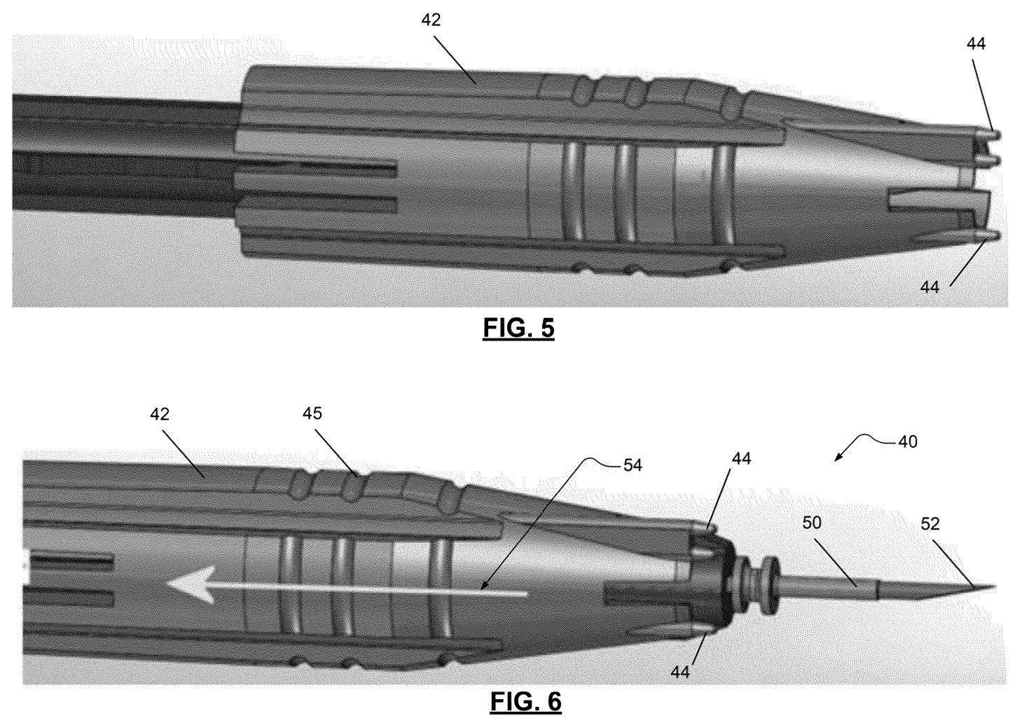

FIG. 5 is a side view of a sheath 42 of an ophthalmic insertion tool 40 in an extended position, and FIG. 6 is a side view of the sheath 42 of FIG. 5 in a retracted position. As shown in FIG. 6, the sheath 42 may be moved in a longitudinal direction 53 to expose a cannula 50 and a trocar tip 52 of the ophthalmic insertion tool 40.

The ophthalmic insertion tool 40 includes multiple scleral markers 44. As shown in FIGS. 5 and 6, each scleral marker 44 may be a protrusion from an end of the sheath 42. Each scleral marker 44 may be positioned at a specific distance with respect to, e.g., other scleral markers 44, an end of the trocar tip 52, an end of the sheath 42, etc. For example, FIGS. 5 and 6 illustrate multiple scleral markers 44 spaced apart from one another along an outer surface of the housing. The scleral markers 44 may be spaced apart from one another by any suitable distance, such as three millimeters, four millimeters, lesser or greater distances, etc.

The housing of the ophthalmic insertion tool 40 may include multiple indentations 45 and protruding ridges on an outer surface of the housing, to assist a surgeon in gripping the housing, etc. Each indentation 45 and protruding ridge extends at least partially around a circumference of the housing, in a direction perpendicular to a longitudinal axis 54 of the housing.

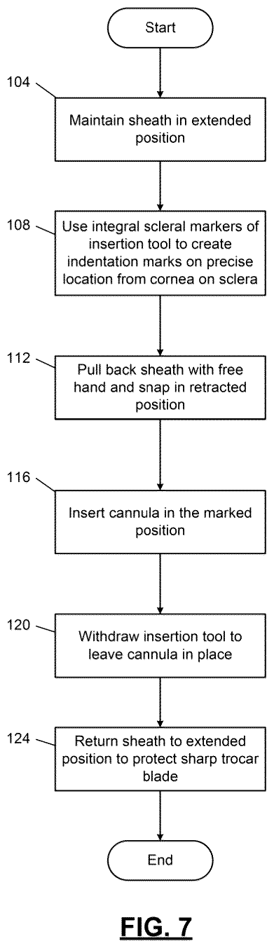

FIG. 7 is a flow chart depicting an example process for using an ophthalmic insertion tool (such as the ophthalmic insertion tool 10 of FIG. 1). At 104, the process begins by maintaining the sheath (e.g., the retractable sheath 22) in an extended position covering the cannula (e.g., the cannula 20).

At 108, integral scleral markers (e.g., the scleral markers 24) of the insertion tool are used to create indentation marks at a precise location (or multiple precise locations) on the sclera. The location(s) may be at a specified distance from the cornea. For example, known distances of the scleral markers, such as three millimeters apart, four millimeters apart, etc., may allow a surgeon to mark the sclera at known distances according to the scleral markers.

At 112, the retractable sheath is pulled back with a free hand, and snapped into a retracted position. For example, the locking point 26 of FIG. 4 may be used to retain the retractable sheath in the retracted position after the sheath is pulled back.

At 116, the cannula is inserted in the marked position. For example, the cannula may be used to puncture the posterior of a patient's eye, at a location previously marked on the sclera using the scleral markers of the ophthalmic insertion tool.

The ophthalmic insertion tool is withdrawn at 120, while leaving the cannula in place. The cannula may allow a surgeon to insert surgical instruments into the eye of the patient through the cannula. The sheath is returned to the extended position at 124, to protect the sharp trocar blade. For example, the surgeon may apply sufficient force to the sheath in a longitudinal direction to overcome the retention force of the locking point of the housing, to move the sheath back into the extended position covering the trocar blade.

In some example embodiments, an ophthalmic insertion tool package includes a container package, and at least one ophthalmic insertion tool (such as the ophthalmic insertion tool 10 of FIG. 1) inside the container package. The ophthalmic insertion tool may include a housing having a distal end and a proximal end, and a retractable sheath coupled with the distal end of the housing. The retractable sheath is configured to move between a first position covering the distal end of the housing and a second position exposing the distal end of the housing.

The ophthalmic insertion tool includes at least one scleral marker integral with the housing. The at least one scleral marker may be located adjacent the distal end of the housing, the proximal end of the housing, or both the distal end and the proximal end. The ophthalmic insertion tool includes at least one cannula inside the container package. The cannula includes a distal tip with at least one port in communication with a lumen extending through the cannula.

The package may be assembled in any suitable manner to assist a surgeon for use during eye surgery. For example, the at least one cannula may be attached to the distal end of the housing of the at least one ophthalmic insertion tool ahead of time, in the package. Alternatively, the at least on cannula may be configured to be loaded into the housing of the at least one ophthalmic insertion tool. The package may include three ophthalmic insertion tools (or more or less), within the same container package.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. An ophthalmic insertion tool comprising:
a housing having a distal end and a proximal end;
a cannula attached to the distal end of the housing, the cannula including a distal tip with at least one port in communication with a lumen extending through the cannula;
a retractable sheath coupled with the distal end of the housing, the retractable sheath configured to move between a first position covering the distal tip of the cannula and a second position exposing the distal tip of the cannula; and
at least one scleral marker integral with the housing, the at least one scleral marker located adjacent the distal end of the housing, the proximal end of the housing, or both the distal end and the proximal end.

2. The ophthalmic insertion tool of claim 1, wherein the at least one scleral marker is located adjacent the distal end of the housing.

3. The ophthalmic insertion tool of claim 1, wherein the at least one scleral marker is located adjacent the proximal end of the housing.

4. The ophthalmic insertion tool of claim 1, wherein the at least one scleral marker includes a first scleral marker located adjacent the proximal end of the housing, and a second scleral marker adjacent the distal end of the housing.

5. The ophthalmic insertion tool of claim 1, wherein the distal end includes a sharp tip configured to puncture a posterior of an eye of a patient.

6. The ophthalmic insertion tool of claim 1, wherein the retractable sheath is configured to move longitudinally along an outer surface of the housing between the first position and the second position.

7. The ophthalmic insertion tool of claim 6, wherein the retractable sheath is configured to retract from the first position to the second position in response to the distal end of the housing pressing against a sclera of an eye of a patient.

8. The ophthalmic insertion tool of claim 7, wherein the housing includes a first locking point configured to maintain the retractable sheath in the second position to expose the distal tip of the cannula to facilitate puncturing the sclera with the distal tip.

9. The ophthalmic insertion tool of claim 8, wherein the housing includes a second locking point configured to maintain the retractable sheath in the first position to protect the distal tip of the cannula from damage.

10. The ophthalmic insertion tool of claim 9, wherein the second locking point is configured to apply a retention force to the retractable sheath to facilitate marking the sclera according to the at least one scleral marker, while the retractable sheath is maintained in the first position.

11. The ophthalmic insertion tool of claim 8, wherein the housing is configured to release the cannula to maintain the cannula in the sclera as the housing is moved away from the eye of the patient.

12. The ophthalmic insertion tool of claim 11, wherein the cannula is configured to facilitate insertion of at least one surgical instrument into the eye of the patient through the cannula.

13. The ophthalmic insertion tool of claim 1, wherein the at least one scleral marker includes at least one of a protruding tip of an end of the retractable sheath.

14. The ophthalmic insertion tool of claim 13, wherein the protruding tip of the at least one scleral marker is configured to make an indentation when pressed against the sclera of an eye of a patient without incising the sclera.

15. The ophthalmic insertion tool of claim 13, wherein the at least one scleral marker includes multiple scleral markers spaced apart from one another at the end of the retractable sheath.

16. The ophthalmic insertion tool of claim 15, wherein two of the multiple scleral markers are spaced apart from one another by three millimeters or four millimeters.

17. An ophthalmic insertion tool package comprising:
a container package;
at least one ophthalmic insertion tool inside the container package, the ophthalmic insertion tool including,
a housing having a distal end and a proximal end,
a retractable sheath coupled with the distal end of the housing, the retractable sheath configured to move between a first position covering the distal end of the housing and a second position exposing the distal end of the housing, and
at least one scleral marker integral with the housing, the at least one scleral marker located adjacent the distal end of the housing, the proximal end of the housing, or both the distal end and the proximal end; and
at least one cannula inside the container package, the cannula including a distal tip with at least one port in communication with a lumen extending through the cannula.

18. The ophthalmic insertion tool package of claim 17, wherein the at least one cannula is attached to the distal end of the housing of the at least one ophthalmic insertion tool.

19. The ophthalmic insertion tool package of claim 17, wherein the at least on cannula is configured to be loaded into the housing of the at least one ophthalmic insertion tool.

20. The ophthalmic insertion tool package of claim 17, wherein the at least one ophthalmic insertion tool includes at least three ophthalmic insertion tools inside the container package.
